(19) United States
(12) Patent Application Publication
(10) Pub. No.: US 2007/0225745 A1
(43) Pub. Date: Sep. 27, 2007

(54) Methods and Apparatus for Treating Uterine Fibroids

(75) Inventors: Kevin R. Arnal, Excelsior, MN (US); Kimberly A. Anderson, Eagan, MN (US)

Correspondence Address: AMS RESEARCH CORPORATION, 10700 BREN ROAD WEST, MINNETONKA, MN, 55343, US

(73) Assignee: AMS RESEARCH CORPORATION, Minnetonka, MN (US)

(21) Appl. No.: 11/688,479

(22) Filed: Mar. 20, 2007

Related U.S. Application Data
(60) Provisional application No. 60/784,304, filed on Mar. 21, 2006.

(51) Int. Cl. A61M 29/00

(52) U.S. Cl. 606/192

(57) ABSTRACT

Minimally invasive surgical instruments and procedures for treating fibroids within avascular space, particularly uterine fibroids, by interrupting the blood supply to the fibroid, are disclosed. One embodiment of a dissection instrument for detaching the fibroid comprises fluid delivery into the avascular space to expand the space and detach the fibroid mass from blood vessels traversing the avascular space through hydro-dissection. The dissection instrument may comprise an inflatable balloon that is shaped to be inflated in the avascular space to expand and dissect the space, at least partially surrounding the fibroid mass, and detach the blood vessels traversing the avascular space. The dissection instrument may comprises a fibroid mass penetrating and gripping tip that enables twisting or rotation of the fibroid mass sufficiently to detach the blood vessels. The detached fibroid mass may be left in place or removed.

### RELATED APPLICATION

This application claims priority to U.S. Provisional Application Ser. No. 60/784,304 filed Mar. 21, 2006, the entire content of which is incorporated herein by reference.

### TECHNICAL FIELD

The present invention pertains to minimally invasive surgical instruments and procedures for treating fibroids, particularly uterine fibroids, by interrupting the blood supply to the fibroid.

### BACKGROUND

Uterine fibroids are noncancerous growths of the uterus that are common in women and are not associated with an increased risk of uterine cancer and almost never develop into cancer. Uterine fibroids, also called fibromyomas, leiomyomas or myomas, develop from the smooth muscular tissue of the uterus (myometrium). A single cell reproduces repeatedly, eventually creating a pale, firm, rubbery tissue mass distinct from surrounding or neighboring normal myometrium. Fibroids range in size from seedlings, undetectable by the human eye, to bulky masses that can distort and enlarge the uterus. They can be single or multiple, in extreme cases expanding the uterus so much that it reaches the rib cage.

Uterine fibroids are clinically apparent in 20% to 25% of women during the reproductive years and cause symptoms necessitating treatment, typically surgical removal of the uterus. Such signs and symptoms are influenced by fibroid size and location and include heavy or prolonged menstrual bleeding, pelvic pressure or pain, urinary incontinence, frequent urination or urine retention. Fibroids that grow into the inner cavity of the uterus (submucosal fibroids) are believed primarily responsible for prolonged, heavy menstrual bleeding. Fibroids that project to the outside of the uterus (subserosal fibroids) can press on the bladder or ureters, causing urinary symptoms. Subserosal fibroids that bulge from the posterior of the uterus can press either on the rectum, causing constipation, or on spinal nerves, causing backache. A fibroid that hangs by a stalk inside or outside the uterus (pedunculated fibroid) can trigger pain by turning on its stalk. Fibroids that are intermediate the inner cavity and the outer surface of the uterus (intramural fibroids) may also be symptomatic. Exemplary intramural, submucosal, subserosal, and pedunculated fibroids are illustrated in FIG. 1 as described below.

Preferred treatments of heavy menstrual bleeding and pelvic pressure include administration of medications that target hormones regulating menstrual cycle, which may shrink but not eliminate fibroids. Hysterectomies (surgical removal of the uterus) are performed to treat non-childbearing women exhibiting symptoms that cannot be alleviated by medications. The surgical removal of the uterus requires exposing it sufficiently, ligating and severing the arteries and Fallopian tubes, severing the broad ligament and other ligaments from the uterine body, and severing the cervix from the fornix. Thus, in addition to the loss of reproductive capability, a hysterectomy requires major invasive surgery that can involve excessive blood loss, prolonged convalescence, attendant pain and discomfort, and economic costs. Newer treatment methods have been developed or proposed for at least some of these diseases and conditions. Nevertheless, hysterectomy remains the treatment of choice to treat the conditions and diseases listed above while less drastic treatments continue to be explored.

Uterine fibroids are highly vascularized and encapsulated such that an avascular space separates the distinct outer encapsulating surface of the fibroid from normal myometrium. The normal myometrium is also well vascularized, and a more limited number of a blood vessels cross the avascular space to nourish the fibroid. Thus, it has been observed that a uterine fibroid can be readily excised from the avascular space after the uterus is surgically exposed in a myomectomy procedure. However, the surgical exposure of the uterus through the abdominal wall in a myomectomy procedure can involve risks and complications, and fibroids may recur over time ultimately necessitating a repeated myomectomy or ultimately a hysterectomy.

Consequently, less invasive procedures have been proposed to shrink or remove fibroids. Fast growing fibroids require more oxygenated blood than normal myometrium. Consequently fibroid growth can be halted and reversed by blocking uterine artery blood flow in a minimally invasive uterine artery embolization procedure, which reduces the oxygenated blood supply to the uterus. This procedure involves some risk of ischemia of the normal myometrium and ovaries. It has been proposed to surgically expose the uterine arteries and to ligate or clamp the uterine arteries or to non-surgically apply clamps to the uterine arteries long enough to cause fibroid cell death without permanently reducing arterial blood flow.

It has also been proposed in U.S. Pat. No. 6,059,766 to access a fibroid to be treated employing a minimally invasive catheter or probe to deliver the embolyzing material directly into vessels of the fibroid mass. However, it would appear to be difficult to identify, isolate and penetrate such minute vessels to inject the embolyzing material therein.

Various myolysis procedures, e.g., that disclosed in U.S. Pat. No. 5,979,453, have been developed to deliver electrical current into the fibroid mass via an energy catheter or probe. Cryomyolysis procedures have also been developed to direct a catheter or probe tip cooled with liquid nitrogen into the fibroid mass to effectively freeze the fibroid. Still other approaches involve non-invasive application of MRI guided focused ultrasound energy into the fibroid mass to ablate the tissue and vessel cells. In these procedures and the above described embolyization procedures, the cells of the fibroid are killed in situ and absorbed by the body.

Other minimally invasive procedures and instruments have been proposed to access and core or debulk or fragment and suction out at least a portion of a uterine fibroid as described in U.S. Pat. Nos. 5,290,303, 5,488,958, 5,863,294, 6,032,673, and 6,468,228.

Notwithstanding these advances, a need remains for a simple, minimally invasive instrument and procedure for accessing and removing a uterine fibroid from the avascular space

### SUMMARY

The preferred embodiments of the present invention incorporate a number of inventive features that address the above-described problems that may be combined as illustrated by the preferred embodiments or advantageously separately employed.

In preferred embodiments, a uterine fibroid is accessed and the avascular space surrounding the uterine fibroid is invaded to effect dissection of the uterine fibroid from the myometrium.

In one preferred embodiment, a uterine fibroid is accessed and the avascular space surrounding the uterine fibroid is invaded to detach the fibroid mass from blood vessels traversing the avascular space to thereby interrupt nourishment of the cells of the fibroid. Various minimally invasive instruments are envisaged that are guided in a minimally invasive pathway from an abdominal incision or a trans-vaginal incision or through a trans-cervical route employing imaging equipment to dispose an instrument distal end or tip proximate a targeted fibroid, advance the instrument distal tip through the surrounding tissue layer(s) into the avascular space, and detach the fibroid within the avascular space from blood vessels feeding the fibroid.

One embodiment of an instrument for detaching the fibroid comprises fluid delivery into the avascular space to expand the space and detach the fibroid mass from blood vessels traversing the avascular space through hydro-dissection. The fluid may comprise one or mixtures of sterile saline, vasorestrictors, blood clotting agents, and embolyzing agents that expand the avascular space and also constrict or occlude or sever the blood vessels traversing the avascular space.

One refinement of the instrument for detaching the fibroid comprises an inflatable balloon that is shaped to be inflated in the avascular space to expand and dissect the space, at least partially surrounding the fibroid mass, and detach the blood vessels traversing the avascular space.

A further refinement of the instrument for detaching the fibroid comprises a fibroid mass penetrating and gripping tip that enables twisting or rotation of the fibroid mass by rotation of the instrument proximal end (or a shaft extending through the instrument body to the gripping end) sufficiently to detach the blood vessels traversing the avascular space from the fibroid mass.

The detached fibroid may be left in place to be absorbed or a macerator or morcellator may be introduced through the same pathway to fragment, macerate or morsellize the fibroid mass and suction the fragments and fluid from the avascular space.

Advantageously, the minimally invasive instruments and procedures minimize patient trauma and procedure time while ensuring safe and reliable

This summary of the invention has been presented here simply to point out some of the ways that the invention overcomes difficulties presented in the prior art and to distinguish the invention from the prior art and is not intended to operate in any manner as a limitation on the interpretation of claims that are presented initially in the patent application and that are ultimately granted.

### BRIEF DESCRIPTION OF THE DRAWINGS

These and other advantages and features of the present invention will be more readily understood from the following detailed description of the preferred embodiments thereof, when considered in conjunction with the drawings, in which like reference numerals indicate like structures throughout the several views, and wherein:

FIG. 1 is a schematic partial cross-section view of the female uterus and vagina illustrating intramural, subserosal, and submucosal uterine fibroids within the myometrium, and pedunculated uterine fibroids extending on pedestals from the myometrium in relation to the ovaries and three minimally invasive approaches to such uterine fibroids;

FIG. 2 is a schematic cross-section view of the mass of a uterine fibroid encapsulated within an avascular space in the myometrium with blood vessels feeding the fibroid crossing the avascular space;

FIGS. 3A-3C schematically illustrate one exemplary process of expanding the avascular space to dissect it and detach the fibroid mass and sever blood vessels crossing the avascular space;

FIGS. 4A-4C schematically illustrate a further exemplary process of expanding the avascular space to dissect it and detach the fibroid mass and sever blood vessels crossing the avascular space;

FIGS. 5A-5C schematically illustrate a still further exemplary process of expanding the avascular space to dissect it, detach the fibroid mass and sever blood vessels crossing the avascular space, and remove the fibroid mass

FIGS. 6A-6C schematically illustrate a still further exemplary process of dissecting the uterine space to detach the fibroid mass and sever blood vessels traversing the avascular space

It will be understood that the drawing figures are not necessarily to scale.

### DETAILED DESCRIPTION OF EXEMPLARY PREFERRED EMBODIMENTS

In the following detailed description, references are made to illustrative embodiments of methods and apparatus for carrying out the invention. It is understood that other embodiments can be utilized without departing from the scope of the invention. Preferred embodiments for minimally invasive surgical instruments and procedures for treating fibroids, particularly uterine fibroids, by isolating or excising fibroid masses are described.

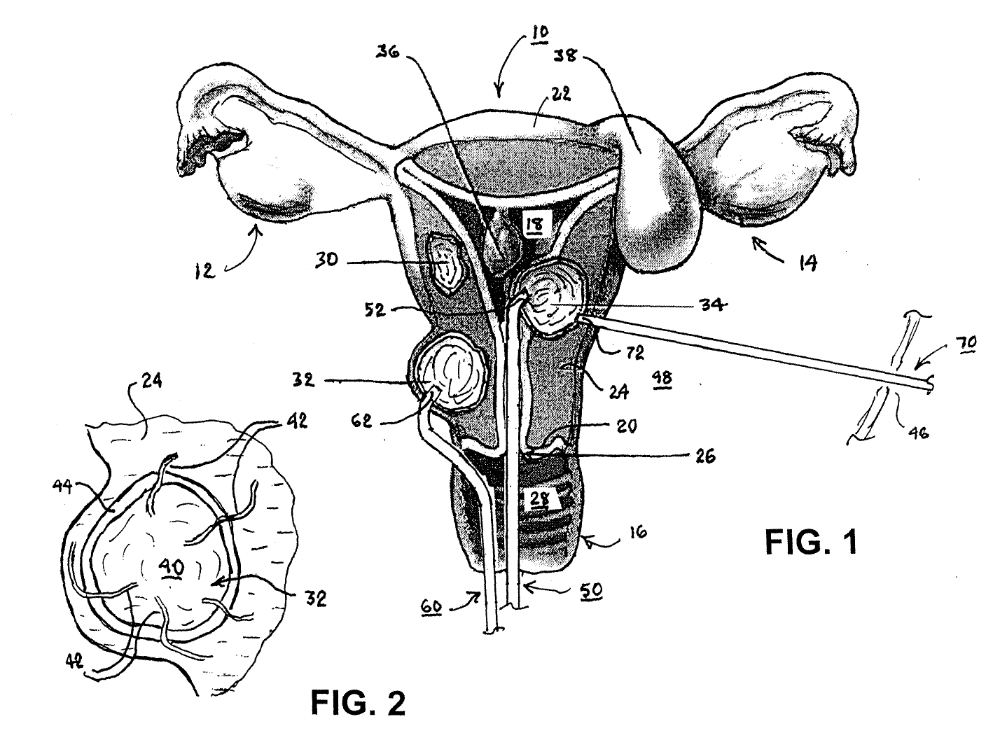

A variety of uterine fibroids are illustrated in FIG. 1 in relation to the female uterus 10, ovaries 12 and 14, and vagina 16 joined with the uterine cavity 18 at the uterine neck or cervix 20. The uterus 10 has a pear-shaped, uterine body extending between a fundus 22 extending right and left to junctions with the right and left Fallopian tubes joined with ovaries 12 and 14 and to the cervix 20 that extends to the vagina 16. The uterus 10 is formed of a smooth muscle uterine wall or myometrium 24 bounded by an outer surosa membrane and an interior uterine mucosa (endometrium) that lines the uterine cavity 18. The uterine body is supported within the pelvis by right and left ligamentous structures such that the uterine body (fundus) is bent (anteflexed) and tilted (anteverted) anteriorly over the bladder and separated from the sacrum by the bowel. The uterine cervix 20 extends into a tissue cul-de-sec of the vagina 16 such that a flexible, annular trough (fornix) of the vagina 16 surrounds and is integrally connected with the cervix 20. The cervical canal 26 joins the uterine cavity 16 with the vaginal cavity 28

A number of uterine fibroids as described above are illustrated in FIG. 1 including an intramural uterine fibroid 30, a subserosal uterine fibroid 32, and a submucosal uterine fibroid 34 generally within the myometrium 24, and pedunculated uterine fibroids 36 and 38 extending on pedestals from the myometrium 24. The subserosal uterine fibroid 32 is located just under the uterine serosa and may be attached to the corpus by a narrow or a broad base. The intramural uterine fibroid 30 is within the thick myometrium 24 and may distort the uterine cavity 18 or cause an irregular external uterine contour. The submucosal uterine fibroid 34 is located within the myometrium 24 proximate the endometrium such that it bulges the endometrium into the uterine cavity 18. The pedunculated uterine fibroid 36 extends into the uterine cavity 18, whereas the pedunculated uterine fibroid 38 extends into the available space outside the myometrium 24.

Uterine fibroids, e.g., the subserosal uterine fibroid 32 depicted in the expanded view of FIG. 2 are well circumscribed, solid, and typically benign fibroid masses 40 or tumors composed of smooth muscle cells and extracellular matrix. Discrete blood vessels 42 (arteries and veins) extending from the myometrium 24 traverse the avascular space 44 to nourish the cells of the fibroid mass 40. In a conventional myomectomy described above, the myometrium 24 is cut to expose the avascular space 44 so that the fibroid mass 40 may be extracted intact or macerated or morsellized and suctioned out of the avascular space 44. In the above-referenced '766 patent, embolyzing material is introduced into the fibroid mass 40 and/or avascular space 44 in an effort to embolyze blood vessels 42 of the fibroid mass. However, introducing the embolyzing material into the minute blood vessels 42 would appear to be difficult to achieve.

By contrast, in accordance with the present invention, the blood vessels 42 traversing the avascular space 44 are detached from the fibroid mass 40 to interrupt blood flow to the fibroid mass 40 causing it to shrink and be absorbed. Returning to FIG. 1, minimally invasive, tubular, dissection instruments 50, 60, 70 can be introduced in a variety of pathways through the myometrium 24 and into the avascular space surrounding a uterine fibroid and/or into the fibroid mass.

The dissection instruments 50, 60, 70 extend from an instrument body distal end adapted to be disposed through a chosen pathway in operative relation to a targeted uterine fibroid through manipulation of the instrument body proximal end outside the patient's body. The advancement and disposition of the instrument body distal end 52, 62, 72 in operative relation to a target uterine fibroid may be guided by fluoroscopy or radio frequency or Doppler ultrasound instrumentation in a manner well known in the art. Additionally or alternatively, the medical instrument 50, 60, 70 may itself incorporate illumination and optical visualization capabilities like an endoscope or laparoscope or Doppler ultrasound imaging capabilities that aid in advancement and proper disposition of the instrument body distal end 52, 62, 72. Preferably, the elongated dissection instrument 50, 60, 70 possesses at least a deflectable distal tip and preferably deflectable instrument body segments in order to manipulate the instrument body to track a curved or convoluted pathway. Other aspects of selected elongated dissection instruments are described below.

For example, the elongated dissection instrument 50 is advanced in a transvaginal approach or pathway through the vaginal cavity 28 and cervical canal 26 into the uterine cavity 18 to dispose the instrument body distal end 52 through the surrounding myometrium 24 and into operative relation with the submucosal uterine fibroid 34. It will be understood that the elongated dissection instrument 50 could alternatively be advanced in the transvaginal approach through the vaginal cavity 28 and cervical canal 26 into the uterine cavity 18 to dispose the instrument body distal end 52 through the surrounding myometrium 24 and into operative relation with the intramural uterine fibroid 30 or the pedunculated uterine fibroid 36 or the subserosal uterine fibroid 32.

The illustrated elongated dissection instrument 60 is advanced in a transcervical approach or pathway through the vaginal cavity 28, then through the fornix of the vagina 16 where it joins the cervix 26, into the body cavity surrounding the uterus 10, and then through the myometrium 24 surrounding the subserosal uterine fibroid 32 to dispose the instrument body distal end 62 into operative relation with the subserosal uterine fibroid 32. It will be understood that the elongated dissection instrument 60 could alternatively be advanced in the transcervical approach to dispose the instrument body distal end 62 through the surrounding myometrium 24 and into operative relation with the intramural uterine fibroid 30 or the pedunculated uterine fibroid 38 or the submucosal uterine fibroid 34.

The illustrated elongated dissection instrument 70 is advanced in an abdominal approach or pathway through the abdominal cavity, via a skin incision 46, to the body cavity 48 surrounding the uterus 10, and then through the myometrium 24 surrounding the submucosal uterine fibroid 34 to dispose the instrument body distal end 72 into operative relation with the submucosal uterine fibroid 34. It will be understood that the elongated dissection instrument 70 could alternatively be advanced in an abdominal approach to dispose the instrument body distal end 72 through the surrounding myometrium 24 and into operative relation with the intramural uterine fibroid 30 or the pedunculated uterine fibroid 38 or the subserosal uterine fibroid 32.

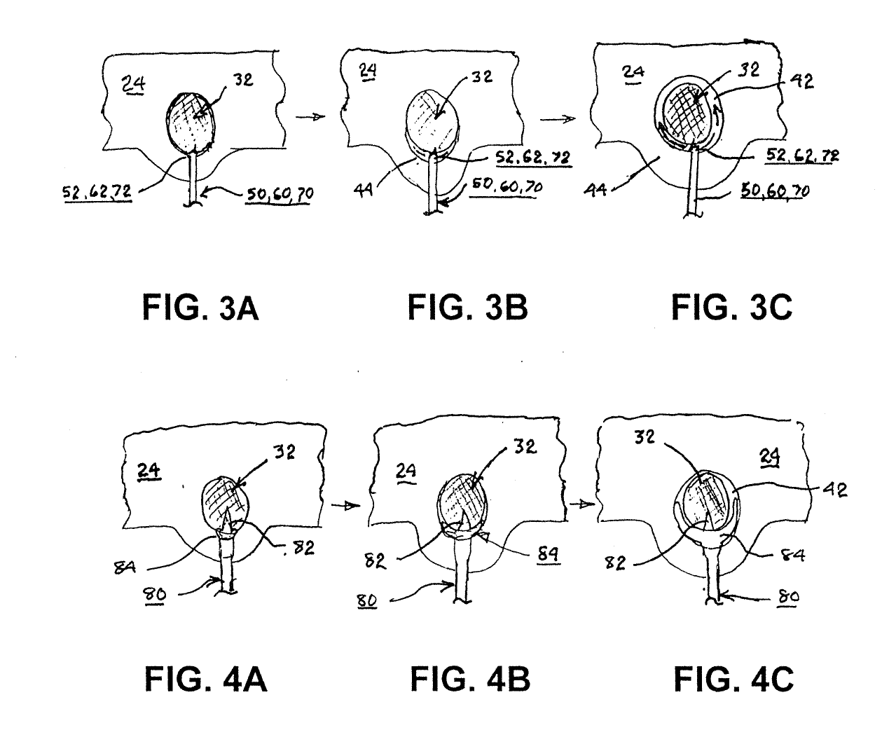

In a first preferred procedure illustrated in FIGS. 3A-3C, one of the elongated dissection instruments 50, 60, 70 having a fluid delivery lumen is introduced through one of the above described pathways into subserosal uterine fibroid 32, for example, fluid is introduced into the avascular space 42, and the avascular space 42 is expanded to stretch and detach the blood vessels (not shown), via hydro-dissection, from the surrounding myometrium 24. The fluid may comprise one or mixtures of sterile saline, vasorestrictors, blood clotting agents, and embolyzing agents that expand the avascular space and also constrict or occlude or sever the blood vessels 44 traversing the avascular space 42.

In FIG. 3A, the elongated dissection instrument distal end 52, 62, 72 is advanced through the myometrium 24 into the avascular space 42. The fluid is delivered as illustrated in FIG. 3B until the avascular space 42 is so expanded as to detach the blood vessels traversing the avascular space 42 as illustrated in FIG. 3C. The elongated dissection instrument 80 is then retracted through the pathway, leaving the fibroid mass to expire in place due to ischemia and be absorbed.

In a second preferred procedure illustrated in FIGS. 4A-4C, an elongated dissection instrument 80 is illustrated having a distal tip 82 optionally adapted to penetrate the fibroid mass of the subserosal uterine fibroid 32, for example, and an expandable balloon 84 adapted to be expanded by fluid delivered through a lumen of the instrument 80 to at least partially expand and fill the avascular space 42 to detach the fibroid mass of the subserosal uterine fibroid 32. The balloon 84 is preferably shaped to expand distally of the instrument distal tip 82 in a cup shape to encircle the fibroid mass of the uterine fibroid. The elongated dissection instrument 80 can be adapted to follow any of the pathways of the above-described instruments 50, 60, 70 to access any of the intramural uterine fibroid 30 or the pedunculated uterine fibroids 36, 38 or the submucosal uterine fibroid 34. In FIG. 4A, the elongated dissection instrument distal end 82 is advanced, with balloon 84 deflated, through the myometrium 24 into the avascular space 42 and optionally into the fibroid mass. The fluid is delivered into the balloon 84 as illustrated in FIG. 4B and 4C until the avascular space 42 is so expanded and dissected by the expanded balloon 84 as to detach the blood vessels traversing the avascular space 42. Then, the balloon 84 is deflated to enable withdrawal of the elongated dissection instrument 80 through the pathway, leaving the fibroid mass to expire in place due to ischemia and be absorbed.

It will be understood that the distal end 82 may be configured with a hook or prongs or screw that may be inserted deeply into the fibroid mass of the uterine fibroid to facilitate retracting a relatively small fibroid mass through the pathway upon withdrawal of the elongated dissection instrument 80.

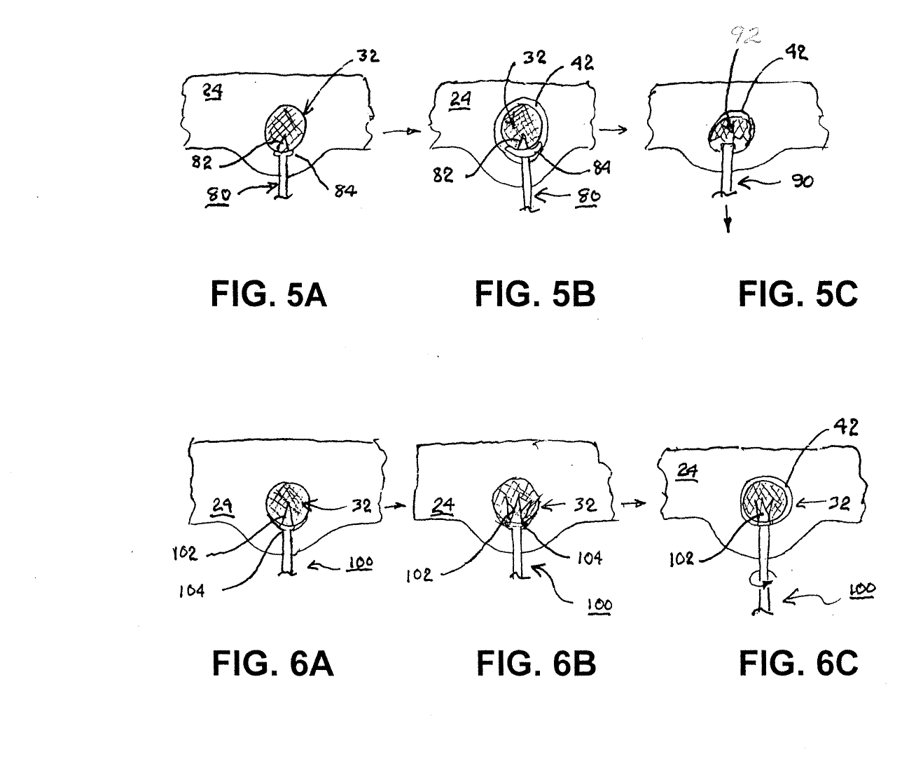

A further variation on the procedure of FIGS. 4A-4C is depicted in FIGS. 5A-5C. In this variation, the avascular space 42 is dissected as summarily illustrated in FIGS. 5A and 5B in the manner described above with respect to FIGS. 4A-4C. The balloon 84 is deflated to enable withdrawal of the elongated dissection instrument 80 through the pathway, and a further elongated fibroid removal instrument 90 is advanced through the pathway to dispose its distal tip 92 adjacent the fibroid mass. The elongated fibroid removal instrument 90 may comprise an aspirator to suction the fibroid mass from the avascular space. Or, the elongated fibroid removal instrument 90 may further include a coring or macerating or morsellizing distal tip to core or pulverize or fragment the fibroid mass into smaller parts under visualization. A suitable instrument may comprise the VersaCut Tissue Morcellator System available from Lumenis, Inc., Santa Clara, Calif. or the Hysteroscopic Morcellation System available from Smith & Nephew Endoscopy, Andover, Mass.

Saline may be injected through an injection lumen of the fibroid removal instrument 90 to aid in aspiration through an aspiration lumen of the instrument 90. It will be understood that features of the elongated dissection instrument 80 and fibroid removal instrument 90 may be combined to form a single elongated dissection instrument to perform the fibroid mass dissection and removal functions, and visualization may be accomplished employing Doppler ultrasound probes and instrumentation.

It may be desirable to provide a mechanism for and process of gripping and twisting the fibroid mass of a uterine fibroid within its avascular space to rotate it sufficiently to detach the blood vessels traversing the avascular space so that the uterine fibroid may expire in place or be removed as described above. An elongated dissection instrument 100 is depicted in FIGS. 6A-6C that can accomplish this. The elongated dissection instrument 100 possesses a gripping head 102 at the instrument distal end and preferably but not necessarily incorporates an inflatable balloon 104 adapted to be selectively inflated by fluid introduced through a balloon inflation lumen. The gripping head 102 is preferably coupled to a gripping head shaft extending through a lumen of the elongated dissection instrument 100. Preferably, gripping elements of the gripping head 102 are deployable into a closed or inactive position enabling introduction or withdrawal through a pathway into a fibroid mass. The gripping elements of the gripping head 102 are then selectively deployable through the gripping head shaft to a deployed position to penetrate, catch and grip the fibroid mass of the subserosal uterine fibroid 32, for example. The elongated dissection instrument 100 can be adapted to follow any of the pathways of the above-described instruments 50, 60, 70 to access any of the intramural uterine fibroid 30 or the pedunculated uterine fibroids 36, 38 or the submucosal uterine fibroid 34.

In FIG. 6A, the gripping head 102, in the closed position, is inserted into the fibroid mass of the subserosal uterine fibroid 32. Gripping elements of the gripping head 102 are deployed into the fibroid mass as shown in FIG. 6B. Then, the gripping head shaft or the entire length of the elongated dissection instrument 100 is rotated or twisted back and forth as shown in FIG. 6C to dissect the avascular space 42 and detach the blood vessels nourishing the fibroid mass. The balloon 104 (if provided) may be selectively inflated and deflated to aid in expansion of the avascular space 42. Alternatively, fluids may be introduced into the avascular space 42 through a fluid delivery lumen of the elongated dissection instrument 100.

The detached fibroid 32 may be left in place to be absorbed or the instrument 90 of FIG. 5C may be employed as described above to remove the fibroid mass fragments and fluid from the avascular space 42. It will again be understood that features of the elongated dissection instruments 100 and fibroid removal instrument 90 may be combined to form a single elongated dissection instrument to perform the fibroid mass dissection and removal functions, and visualization may be accomplished employing Doppler ultrasound probes and instrumentation.

It will also be understood that the above-described elongated dissection instruments 50, 60, 70, 80, 90, 100 may be introduced by themselves or through the lumen of a guiding instrument advanced through the above-described pathways.

All patents and publications referenced herein are hereby incorporated by reference in their entireties.

It will be understood that certain of the above-described structures, functions and operations of the above-described preferred embodiments are not necessary to practice the present invention and are included in the description simply for completeness of an exemplary embodiment or embodiments. It will also be understood that there may be other structures, functions and operations ancillary to the typical surgical procedures that are not disclosed and are not necessary to the practice of the present invention.

In addition, it will be understood that specifically described structures, functions and operations set forth in the above-referenced patents can be practiced in conjunction with the present invention, but they are not essential to its practice.

It is therefore to be understood, that within the scope of the appended claims, the invention may be practiced otherwise than as specifically described without actually departing from the spirit and scope of the present invention.

1. A method of treating uterine fibroids within an avascular space in the myometrium of the uterus, comprising:
introducing the distal end of a fibroid dissection instrument into the avascular space surrounding a uterine fibroid;
expanding the avascular space between the uterine fibroid and the myometrium;
detaching blood vessels extending between the uterine fibroid and the myometrium.

2. The method of claim 1, further comprising:
introducing an elongated fibroid removal instrument into the dissected uterine fibroid; and
operating the fibroid removal instrument to physically remove the detached uterine fibroid from the myometrium.

3. The method of claim 2, wherein the expanding step further comprises:
delivering fluid through the fibroid dissection instrument into the avascular space surrounding the uterine fibroid to expand the space and detach the fibroid mass from blood vessels traversing the avascular space through hydro-dissection.

4. The method of claim 2, wherein the fibroid dissection instrument comprises an inflatable balloon at the distal end of the fibroid dissection instrument, and:
the introducing step comprises introducing the inflatable balloon at the distal end of the fibroid dissection instrument into the avascular space surrounding a uterine fibroid; and
the expanding step further comprises inflating the balloon in the avascular space to expand and dissect the space, at least partially surrounding the fibroid mass, and to detach the blood vessels traversing the avascular space.

5. The method of claim 2, wherein the fibroid dissection instrument comprises a fibroid mass penetrating and gripping tip at the distal end of the fibroid dissection instrument, and:
the introducing step comprises introducing the fibroid mass penetrating and gripping tip into the uterine fibroid; and
manipulating the fibroid dissection instrument sufficiently to detach the uterine fibroid from blood vessels traversing the avascular space.

6. The method of claim 1, further comprising:
introducing an elongated morcellator into the dissected uterine fibroid; and
operating the morcellator to morsellize the dissected uterine fibroid.

7. The method of claim 1, further comprising:
introducing an elongated morcellator into the dissected uterine fibroid;
operating the morcellator to morsellize the fibroid into morsels; and
suctioning the morsels from the avascular space.

8. The method of claim 1, wherein the introducing step further comprises:
introducing the fibroid dissection instrument through a minimally invasive pathway from one of an abdominal incision or a trans-vaginal incision or through a trans-cervical route employing imaging equipment to dispose the instrument distal end proximate a targeted fibroid; and
advancing the instrument distal tip through the surrounding myometrium into the avascular space.

9. The method of claim 1, wherein the expanding step further comprises:
delivering fluid through the fibroid dissection instrument into the avascular space surrounding the uterine fibroid to expand the space and detach the fibroid mass from blood vessels traversing the avascular space through hydro-dissection.

10. The method of claim 1, wherein the fibroid dissection instrument comprises an inflatable balloon at the distal end of the fibroid dissection instrument, and:
the introducing step comprises introducing the inflatable balloon at the distal end of the fibroid dissection instrument into the avascular space surrounding a uterine fibroid; and
the expanding step further comprises inflating the balloon in the avascular space to expand and dissect the space, at least partially surrounding the fibroid mass, and to detach the blood vessels traversing the avascular space.

11. The method of claim 1, wherein the fibroid dissection instrument comprises a fibroid mass penetrating and gripping tip at the distal end of the fibroid dissection instrument, and:
the introducing step comprises introducing the fibroid mass penetrating and gripping tip into the uterine fibroid; and
manipulating the fibroid dissection instrument sufficiently to detach the uterine fibroid from blood vessels traversing the avascular space.

12. Apparatus for treating uterine fibroids within an avascular space in the myometrium of the uterus, comprising:
a fibroid dissection instrument extending between and instrument proximal end and an instrument distal end adapted to be inserted through a pathway of the body to dispose the instrument distal end in the avascular space surrounding a target fibroid, the fibroid dissection instrument further comprising means for expanding the avascular space between the uterine fibroid and the myometrium to dissect the uterine fibroid and sever blood vessels traversing the avascular space, thereby interrupting blood flow to the fibroid.

13. The apparatus of claim 12, further comprising means for physically removing the detached uterine fibroid from the avascular space.

14. The apparatus of claim 12, further comprising means for morsellizing the detached uterine fibroid into fibroid morsels and physically removing the uterine fibroid morsels from the avascular space.

15. The apparatus of claim 12, further comprising means for morsellizing the detached uterine fibroid into fibroid morsels and suctioning the uterine fibroid morsels from the avascular space.

16. The apparatus of claim 12, wherein the means for expanding the avascular space further comprises means for delivering fluid through the fibroid dissection instrument into the avascular space surrounding the uterine fibroid to expand the space and detach the fibroid mass from blood vessels traversing the avascular space through hydro-dissection.

17. The apparatus of claim 12, wherein the means for expanding the avascular space further comprises:
an inflatable balloon at the distal end of the fibroid dissection instrument adapted to be introduced into the avascular space surrounding a uterine fibroid; and
means for inflating the balloon in the avascular space to expand and dissect the space, at least partially surrounding the fibroid mass, and to detach the blood vessels traversing the avascular space.

18. The apparatus of claim 12, wherein the means for expanding the avascular space further comprises:
a fibroid mass penetrating and gripping tip at the distal end of the fibroid dissection instrument adapted to be advanced into the fibroid mass, and;
means for manipulating the fibroid dissection instrument sufficiently to detach the uterine fibroid from blood vessels traversing the avascular space.

19. Apparatus for treating uterine fibroids within an avascular space in the myometrium of the uterus, comprising:
a fibroid dissection instrument extending between and instrument proximal end and an instrument distal end sized and shaped to be inserted through a minimally invasive pathway from one of an abdominal incision or a trans-vaginal incision or through a trans-cervical route to dispose the instrument distal end in the avascular space surrounding a target fibroid, the fibroid dissection instrument further comprising means for expanding the avascular space between the uterine fibroid and the myometrium to dissect the uterine fibroid and sever blood vessels traversing the avascular space, thereby interrupting blood flow to the fibroid.

20. The apparatus of claim 19, further comprising means for physically removing the detached uterine fibroid from the avascular space.
